(19) United States
(12) Patent Application Publication
(10) Pub. No.: US 2011/0087199 A1
(43) Pub. Date: Apr. 14, 2011

(54) MULTIFUNCTION CARBASUS FOR LAPAROSCOPIC OPERATION

(76) Inventors: Kai PAN, Shenzhen (CN); Xuefei Yang, Shenzhen (CN); Chang Zhu, Shenzhen (CN)

(21) Appl. No.: 12/891,545

(22) Filed: Sep. 27, 2010

(30) Foreign Application Priority Data
Oct. 14, 2009 (CN) 200910207236.1

(51) Int. Cl. A61B 17/00

(52) U.S. Cl. 606/1

(57) ABSTRACT

A special carbasus for laparoscopic operations belongs to surgical instruments for laparoscopic operations. A carbasus is needed to perform many handlings in laparoscopic operations. However, a self-made carbasus strip currently used in clinic is inconvenient in use and has just single function. To solve the above-mentioned problems, the present invention provides a carbasus which is folded in a folding-fan-shaped manner with both ends folded toward inside and fixed to form compact obtuse ends, provided with a tantalum wire having length scales and cut with vertical and horizontal openings, such that the carbasus can be easily put in and taken out through a channel on the abdominal wall conveniently and have functions of measurement, X-ray development, barrier separation, transportation of small specimens and instruments, rope and separators in laparoscopic operations.

The present invention claims the benefit of CN Application No. 200910207236.1 entitled MULTIFUNCTION CARBASUS FOR LAPAROSCOPIC OPERATION, filed with the Chinese State Intellectual Property Office on Oct. 14, 2009. The entire disclosure thereof is incorporated herein by reference. A claim of priority to all, to the extent appropriate is made.

### FIELD OF THE INVENTION

The present invention relates to a laparoscopic surgical instrument, and more specifically, to a multifunction carbasus specially designed for laparoscopic operation and used for intraoperative hemostasia, barrier, measurement, drawing, peeling, and transportation of small specimens or instruments.

### BACKGROUND OF THE INVENTION

At present, the laparoscopic operation has been widely used in clinic. By establishing the channel of the abdominal wall and a pneumoperitoneum, the laparoscopic operation is performed with the laparoscopic instruments through the channel. The carbasus is generally used for hemostasis, exposure and peeling during surgical operation. Differing from the laparotomy operation, it is not allowed in the laparoscopic operation to place the carbasus having a variety of sizes and shapes into the abdominal cavity, instead, a small piece of carbasus may pass through the channel of the abdominal wall having a diameter from 5 mm to 12 mm. However, up to now, there is no special carbasus for the laparoscopic operation. What is mostly used in clinic is a small self-made carbasus roll, which is irregular in shape and has just single function and thus is inconvenient in use.

### SUMMARY OF THE INVENTION

To meet the special requirements of carbasus for laparoscopic operations, the present invention provides a multifunction carbasus for laparoscopic operations, which may be easily put in and taken out through a channel on the abdominal wall in laparoscopic operations and perform many functions such as peeling, barrier and rope, besides the functions same as that played by the conventional carbasus, for example compression hemostasis and dipping oozing blood.

The present invention provides a carbasus for laparoscopic operations including a top layer of carbasus and a bottom layer of carbasus. The carbasus is formed by superposing the top layer of carbasus and the bottom layer of carbasus, and the superposed top layer of carbasus and bottom layer of carbasus are folded into a strip in a folding-fan-like manner. Two compact obtuse ends are formed by folding and fixing both ends of the strip towards inside.

Specifically, the carbasus is formed by superposing two layers of carbasus which have a length of 20 cm and a width of 10 cm. The carbasus is folded into a folding-fan shape (with a fold width of 1 cm) along the longitudinal side thereof, and finally formed into a carbasus strip. Then, both ends of the carbasus are folded towards inside and fixed to form two compact obtuse ends. A blue tantalum wire with length scales is provided on the long edge of one side of the bottom layer of carbasus. Length scales with a unit of 5 cm are marked on the tantalum wire. The tantalum wire is positioned at one side of the carbasus strip after being folded. An opening (vertical opening) of 8 cm is cut at the middle position along a direction perpendicular to the longitudinal axis and a opening (horizontal opening) of 8 cm is cut at the middle position beside this vertical opening along a direction parallel to the longitudinal axis in the top layer of carbasus. A corresponding horizontal opening is cut in the bottom layer of the carbasus too. Accordingly, two horizontal openings penetrate through the two layers of the carbasus. A little flap is provided at the middle position of an edge of the vertical opening. The specific embodiments of the multifunction carbasus are described as follows.

In an embodiment, the carbasus is folded in a folding-fan-shaped manner so as to form a strip. Both ends of the carbasus strip are folded and fixed such that they may be clamped by a laparoscopic instrument and the carbasus strip may be put in and taken out through a channel on the abdominal wall conveniently. The folding-fan-like structure may be easily opened in abdominal cavity by laparoscopic operation instruments, and forms a sheet-shaped absorbing surface after being opened which is used not only to oppress and dip oozing blood but also to fend off slidable abdominal organs such as intestina so as to expose an operative visual field and protect the abdominal organs.

In another embodiment, a tantalum wire is provided on the carbasus to meet the requirement that the medical carbasus should be provided with an X-ray fluoroscopic sign so as to be recovered easily. The length scales may be used for a measuring tool in operation, for example, may be used to measure the length of intestina and measure the diameter of tumors.

In a further embodiment, when the vertical opening of the top layer of carbasus is opened, a pocket is formed between the top and bottom layers of carbasus, into which a small specimen of tissue such as lymph node, or a small instrument may be loaded and which may be put in and taken out from the abdominal cavity through a trocar that is used to build a channel on the abdominal wall in laparoscopic operations. The pocket plays a function of isolation and protection and prevents the small specimens or the small instruments from falling off. A little flap is provided at an edge of the vertical opening to easily open the pocket in abdominal cavity with instruments.

In an additional embodiment, the horizontal openings penetrating through the two layers of carbasus allow the carbasus to function as a rope. When one end of the carbasus strip passes through the horizontal openings, a slipknot rope is formed to catch and draw a tissue such as intestine in various directions, thereby helping to expose the operative visual field and protecting intestina from being harmed.

In a further embodiment, compact obtuse heads may be formed by folding the carbasus and fixed the two ends of the carbasus, which may be used as separators commonly used in surgical operation for performing a safe and fine blunt peeling.

### BRIEF DESCRIPTION OF THE DRAWINGS

FIG. 1 is a schematic plan view of a top layer of carbasus;

FIG. 2 is a schematic plan view of a bottom layer of carbasus;

FIG. 3 shows the folded carbasus;

FIG. 4 is a schematic top view of the formed carbasus;

FIG. 5 is a schematic bottom view of the formed carbasus;

FIG. 6 is a schematic view of one side of the formed carbasus, on which a tantalum wire is provided;

FIG. 7 is a schematic view of the other side of the formed carbasus;

FIG. 8 is a schematic view of the formed carbasus from the top layer thereof when it is unfolded;

FIG. 9 is a schematic view of the formed carbasus from the bottom layer thereof when it is unfolded;

FIG. 10 is a schematic view of the carbasus used as a barrier;

FIG. 11 is a schematic view of the carbasus used as a measuring tool;

FIG. 12 is a schematic view of the carbasus under an X-ray fluoroscopy;

FIG. 13 is a schematic view of the carbasus used as a pocket;

FIG. 14 is a schematic view of the carbasus used as a rope; and

FIG. 15 is a schematic view of the carbasus used as a separator.

### REFERENCE NUMERALS IN FIGURES

| | | |
|---|---|---|
| 1 - vertical opening | 2 - little flap | 3 - horizontal opening; |
| 4 - tantalum wire having scale | | 5 - folded fixed end |

### DETAILED DESCRIPTION OF THE INVENTION

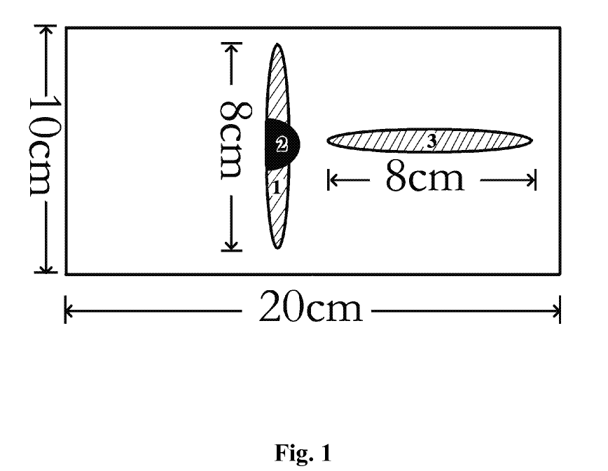

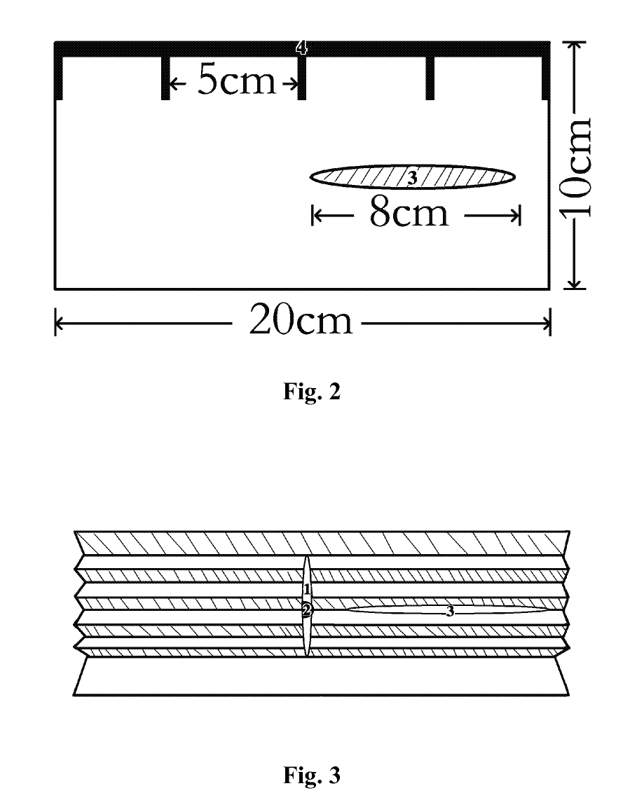

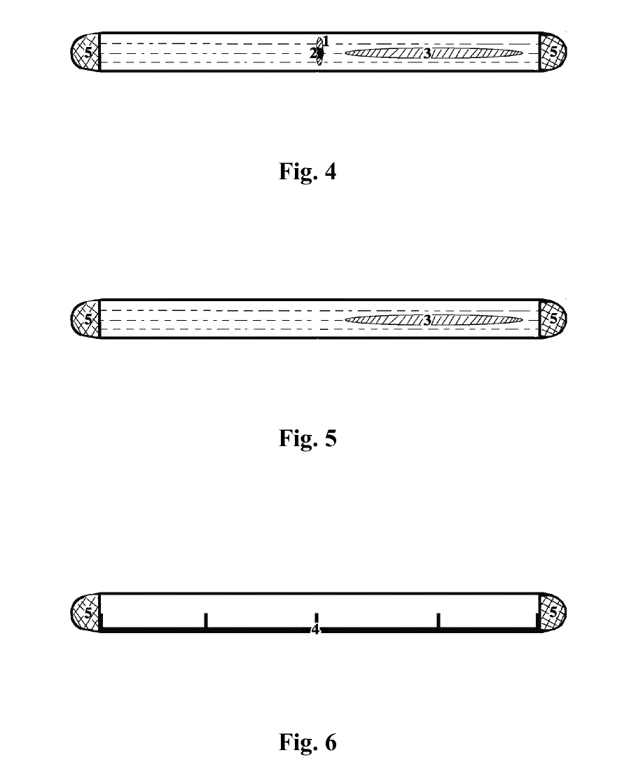

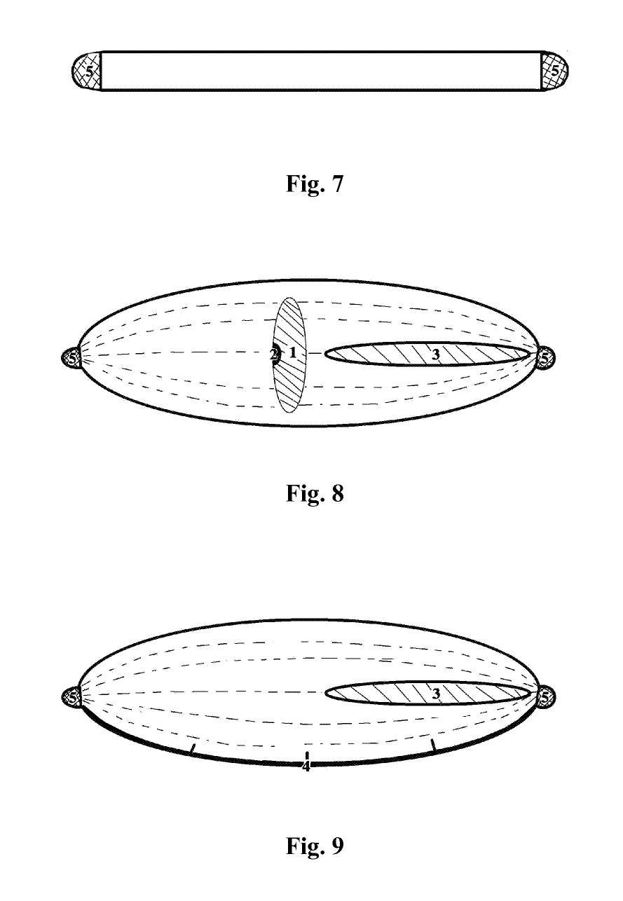

A multifunction carbasus for laparoscopic operations is formed by superposing two layers of carbasus (whose length is 20 cm and width is 10 cm), i.e. the top and bottom layers of carbasus. A vertical opening and a horizontal opening (whose length is 8 cm) are cut at the positions of midlines in the longitudinal direction and the widthwise direction in the top layer of carbasus, respectively (FIG. 1). A horizontal opening corresponding to the horizontal opening in the top layer of carbasus is cut in the bottom layer of carbasus (FIG. 2). The carbasus is folded in a folding-fan-shaped or corrugation-shaped manner along the longitudinal side thereof with a fold width of 1 cm (FIG. 3). The appearance of the carbasus after being folded is shown in FIGS. 4 to 9. Thus, the carbasus folded into a strip may be easily put in and taken out through trocars for laparoscopic operations.

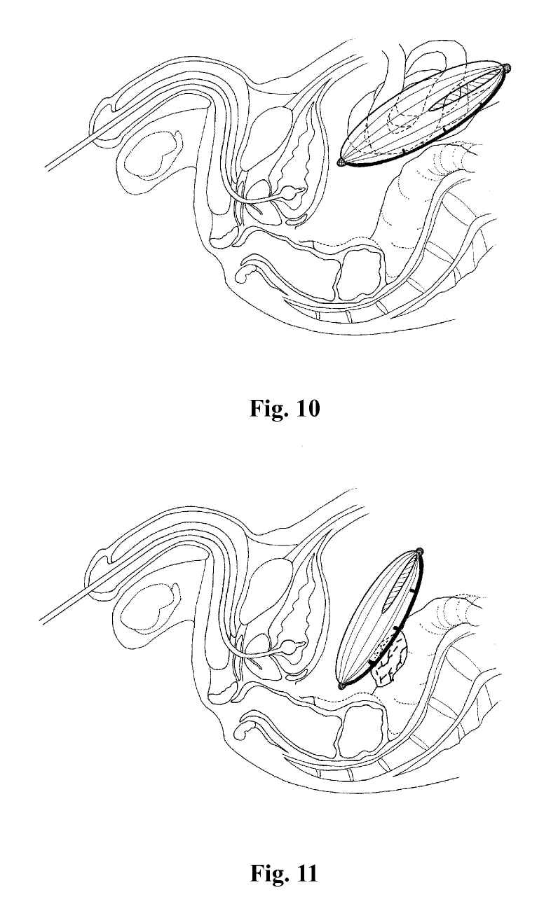

In the embodiment shown in FIG. 10, the carbasus may be unfolded to use as a barrier which can fend off intestines and other organs in the operative visual field to expose the operative visual field and protect abdominal viscera.

In the embodiment shown in FIG. 11, a tantalum wire having length scale (5 cm) may be function as measuring tool in laparoscopic operations, which may be used to determine the size of tumors or other niduses, and may also be used to measure the length of intestina.

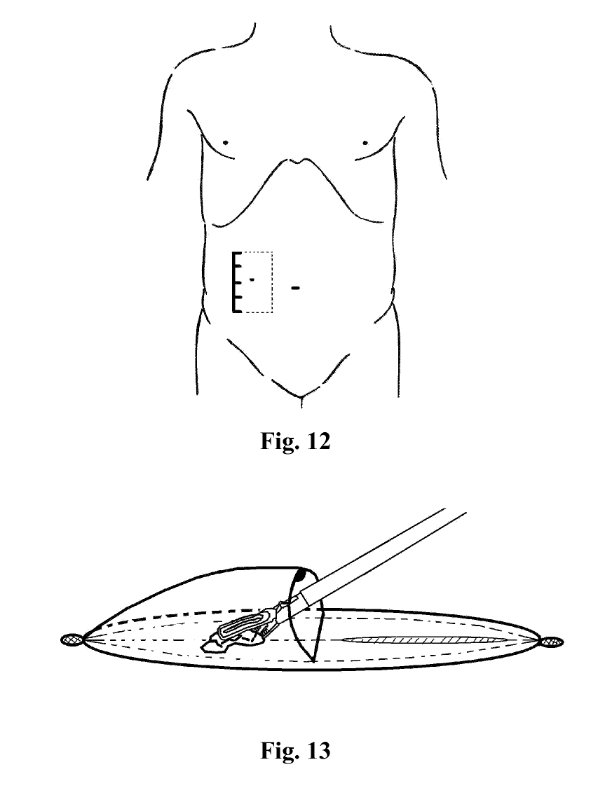

In the embodiment shown in FIG. 12, the tantalum wire provided on the carbasus may be developed under an X-ray fluoroscopy to meet the requirement that a carbasus for surgical operations should be provided with an X-ray fluoroscopic sign so as to facilitate the recovering of the lost carbasus.

In the embodiment shown in FIG. 13, when the vertical opening of the top layer of the carbasus is opened, the top and bottom layers of the carbasus form a pocket, into which small specimens such as lymph node, or small instruments such as a suture needle may be loaded in laparoscopic operations. The pocket not only facilitates putting in and taking out of the carbasus loading with small specimens or instruments through a channel on the abdominal wall but also plays a function of isolation and protection to prevent small specimens or instruments from falling off and being lost. A little flap is provided on one side of the vertical opening to easily open the pocket.

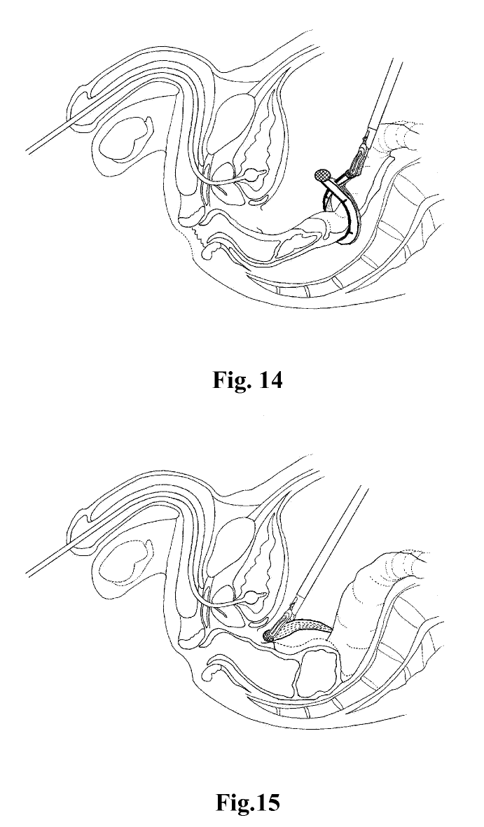

In the embodiment shown in FIG. 14, when one end of the carbasus passes through the horizontal openings of the two layers of the carbasus, a slipknot rope is formed by which intestina may be caught and drawn in various directions. Therefore, it facilitates the exposure of the operative visual field and protects the intestina from being harmed by the instruments.

In the embodiment shown in FIG. 15, after folding the carbasus and fixing both ends of it, compact obtuse heads are formed, which may be used as separators in laparoscopic operations, so as to perform a safe and fine blunt peeling. Thus, it is unnecessary to place another separator into abdominal cavity, so as to avoid the fact that it is difficult to find the small missing separator.

What is claimed is:

1. A carbasus for laparoscopic operations comprising a top layer of carbasus and a bottom layer of carbasus, wherein the carbasus is formed by superposing the top layer of carbasus and the bottom layer of carbasus, the superposed top layer of carbasus and bottom layer of carbasus are folded into a strip in a folding-fan-like manner, and two compact obtuse ends are formed by folding and fixing both ends of the strip towards inside.

2. The carbasus for laparoscopic operations according to claim 1, wherein a first opening is provided on the top layer of carbasus.

3. The carbasus for laparoscopic operations according to claim 2, wherein a flap is provided on an edge of the first opening.

4. The carbasus for laparoscopic operations according to claim 2, wherein the first opening is opened along a direction perpendicular to a longitudinal direction of the strip and located at a middle position in the longitudinal direction of the strip.

5. The carbasus for laparoscopic operations according to claim 1, wherein two second openings are provided on corresponding positions of the top layer of carbasus and the bottom layer of carbasus, respectively.

6. The carbasus for laparoscopic operations according to claim 2, wherein one second opening is provided at a position except the first opening of the top layer of carbasus, and one second opening is also provided at a corresponding position of the bottom layer of carbasus.

7. The carbasus for laparoscopic operations according to claim 5, wherein the second openings are opened along a longitudinal direction of the strip and located at ½ position in a direction perpendicular to the longitudinal direction of the strip.

8. The carbasus for laparoscopic operations according to claim 6, wherein the second openings are opened along a longitudinal direction of the strip and located at ½ position in a direction perpendicular to the longitudinal direction of the strip.

9. The carbasus for laparoscopic operations according to claim 1, wherein a tantalum wire is provided on one side of the carbasus.

10. The carbasus for laparoscopic operations according to claim 9, wherein the tantalum wire is provided with length scales.

11. The carbasus for laparoscopic operations according to claim 2, wherein a tantalum wire is provided on one side of the carbasus.

12. The carbasus for laparoscopic operations according to claim 5, wherein a tantalum wire is provided on one side of the carbasus.

13. The carbasus for laparoscopic operations according to claim 6, wherein a tantalum wire is provided on one side of the carbasus.
